(19) United States
(12) Patent Application Publication
(10) Pub. No.: US 2013/0266676 A1
(43) Pub. Date: Oct. 10, 2013

(54) TERMINALIA CHEBULA COMPOSITIONS AND METHOD OF EXTRACTING SAME

(71) Applicant: NATREON, INC., New Brunswick, NJ (US)

(72) Inventors: Shibnath Ghosal, Kolkata (IN); Muruganandam Veeraragavan, Kolkata (IN); Sanyasi R. Kalidindi, East Brunswick, NJ (US)

(73) Assignee: Natreon, Inc., New Brunswick, NJ (US)

(21) Appl. No.: 13/859,633

(22) Filed: Apr. 9, 2013

Related U.S. Application Data
(60) Provisional application No. 61/622,323, filed on Apr. 10, 2012.

(51) Int. Cl. A61K 36/185

(52) U.S. Cl. CPC A61K 36/185
USPC 424/769

(57) ABSTRACT

An enriched hydrolyzable tannoid blend derived from Terminalia chebula is provided. An optimized aqueous extraction method for Terminalia chebula is provided to maximize the levels of bioactive hydrolyzable tannoids including chebulagic acid, chebulinic acid and other low molecular weight hydrolyzable tannoids. The method produces a Terminalia chebula extract containing a hydrolyzable tannoid blend as an amorphous dry powder. In an embodiment, the Terminalia chebula extract contains 8-25% by weight chebulagic acid, 15-30% chebulinic acid, and 5-45% low molecular weight hydrolyzable tannoids. Potential uses of said enriched hydrolyzable tannoid compositions for antioxidant activity in a human subject are described herein.

This application claims the benefit of earlier filed U.S. Provisional Application No. 61/622,323, filed on Apr. 10, 2012, which is hereby incorporated by reference herein.

### FIELD OF THE INVENTION

The present invention relates to an enriched hydrolyzable tannoid blend derived from Terminalia chebula. The invention further relates to a method for extracting Terminalia chebula to obtain a hydrolyzable tannoid powder enriched with chebulagic acid and/or chebulinic acid. This invention further relates to use of said enriched hydrolyzable tannoid compositions for antioxidant defense in a human subject.

### BACKGROUND

Terminalia chebula Retz. (Combretaceae) has been extensively used in Ayurveda, Unani and Homoeopathic systems of medicines for improvement of different health conditions, e.g., constipation, diarrhoea, ulcers, gastroenteritis, asthma, cough, dyspnea, dyspepsia, hemorrhoids, candidiasis, parasites, malabsorption syndrome, hepatomegaly, vesicular and renal calculi, urinary discharges, tumors, skin diseases, leprosy, intermittent fever, rheumatism, arthritis, gout, neuropathy, paralysis, memory loss, epilepsy, depression, leucorrhea, diabetes, cardiovascular diseases, anorexia, and wounds, among others (See, B. Das, Materia Medica of Ayurveda (New Delhi: B. Jain Publishers, 1991), p. 8; K. R. Kirtikar and B. D. Basu, “Terminalia chebula.” In: Indian Medicinal Plants, (2^{nd} Edn., Allahabad, India: Lolit Mohan Basu Publication, 1935), pp. 1020-23; and P. V. Sharma, Dravya Guna Vigyana (Vol. 2, Varanasi: Chaukhamba Bharati Academy, 1995), pp. 753-58). T. chebula fruit and its different solvent extractives were reported to exhibit hepatoprotective, cardioprotective, antimutagenic/anticarcinogenic, cytoprotective, radioprotective, antioxidant and adaptogenic, antimicrobial, antifungal, antiviral, antiamoebic, immunomodulatory, antidiabetic, wound healing, antispasmodic, and purgative activities in various animal models (S. S. Tasduq, et al., Human and Exp. Toxicol. (2006) 25: 11-18; H. Y. Cheng, et al., Biol. Pharm. Bull. (2003) 26:1331-5; S. Kaur, et al., Mutagen Res. (1998) 419: 169-79; Suthienkul, et al., South-East Asian Journal Trop. Med., Public Health (1993) 24: 751-5; Ahmad, et al., J. Ethnopharmacol. (1998) 62: 183-93; and N. K. Rao, et al., BMC Complement Altern. Med. (2006) 6: 127-32). Clinical studies on T. chebula reported oral cleansing effects achieved by reducing the total bacterial counts and streptococcal counts in saliva samples (A. G. Jagtap, et al., J. Ethnopharmacol. (1999) 68: 299-06). A short term clinical trial on patients with simple constipation reported that T. chebula increases stool formation and has the property of evacuating the bowel completely (V. N. Tripathi, et al., Sachitra Ayur. (1983) 35: 733-40).

Terminalia chebula (T. chebula) is rich in tannoid principles. The chief constituent tannoids in the fruit are chebulinic acid, chebulagic acid, corilagin and a tannoid metabolite, gallic acid (J. Bruneton. Pharmacognosy, Phytochemistry, Medicinal Plants. (Paris, France: Laviosier Publishing, 1995), p. 333). Other minor hydrolyzable tannoids reported in T. chebula include punicalagin, chebulanin, neochebulinic acid, 1,2,3,4,6-penta-O-galloyl-β-D-glucose, 1,6,-di-O-galloyl-D-glucose, casuarinin, 3,4,6-tri-O-galloyl-D-glucose, and terchebulin (L. J. Juang, et al., J. Sep. Sci. (2004) 27: 718-24). One source lists T. chebula as having a tannoids content of about 32% by weight (W. Evans. Trease and Evan's Pharmacology. (14th Ed., W.B. Saunders Co. Pvt. Ltd., 1996), p. 493). Other constituents reported in T. chebula include fructose, amino acids, succinic acid, beta-sitosterol, resin and purgative principles of anthroquinone, sennoside, flavonol glycosides, triterpenoids and coumarin conjugated with gallic acids (E. Creencia, et al., KIMIKA (1996) 12: 1-10).

Chemical constituents isolated from T. chebula may vary considerably in type and/or concentration due to a number of factors, e.g., ecological variation, soil variation, and nutrient variation, as well as variations in the process of extraction.

In view of the above, it would be desirable to provide a potent and therapeutically effective extract of T. chebula in a pharmaceutical or nutraceutical composition having improved properties for the treatment or prevention of diseases. In particular, it would be desirable to provide a therapeutically effective extract of T. chebula suitable for use in connection with the treatment, medication, prevention, and/or symptoms associated with free radical-induced ailments. It would also be desirable to provide an extract of T. chebula for use as a nutritional supplement.

If a way could be found to enhance or enrich the levels of bioactive tannins or tannoids including chebulagic acid, chebulinic acid and other low molecular weight hydrolyzable tannoids in a T. chebula extract, this would represent a valuable contribution to the art. A further valuable contribution to the art would be to provide a standardization of this important Ayurvedic rasayana (used as a revitalizer or rejuvenator).

### SUMMARY OF THE INVENTION

One objective of the present invention is to develop an optimized extraction process to enrich the bioactive contents, namely, chebulagic acid, chebulinic acid and/or low molecular weight hydrolyzable tannoids (LMwHTs) in a T. chebula extract.

It is a further objective of the invention to develop an extraction process for T. chebula which is substantially aqueous. It is another objective of the invention to develop an extraction process for T. chebula which is completely aqueous.

In one embodiment, a T. chebula extract contains about 8-15% by weight chebulagic acid, about 20-25% by weight chebulinic acid and about 20-45% by weight other low molecular weight hydrolyzable tannoids. This embodiment provides a T. chebula enriched tannoid blend composition (TC/enriched tannoid blend).

In another embodiment, a T. chebula extract contains about 8-25% by weight chebulagic acid, about 15-30% by weight chebulinic acid and about 5-45% by weight other low molecular weight hydrolyzable tannoids. This embodiment provides a T. chebula enriched tannoid blend composition (TC/enriched tannoid blend).

In another embodiment, a method of making a Terminalia chebula extract composition is provided comprising a hydrolyzable tannoid blend, including the steps of:

(a) providing fruit pericarp portions of a Terminalia chebula plant;

(b) grinding the fruit pericarp Terminalia chebula plant portions to provide a powder;

(c) extracting the Terminalia chebula fruit pericarp powder with water to provide a Terminalia chebula aqueous extract; and

(d) drying the Terminalia chebula aqueous extract to provide a Terminalia chebula extract as a powder.

In another embodiment, a method of treating or preventing a free radical-induced disease or ailment in an individual includes administering to the individual in need of such treatment a therapeutically effective amount of a T. chebula extract containing about 8-25% by weight chebulagic acid, about 15-30% by weight chebulinic acid, and about 5-45% by weight other low molecular weight hydrolyzable tannoids, wherein the Trolox equivalent antioxidant capacity (TEAC) of the composition is at least about 2000 μmoles of trolox equivalents/gm.

### BRIEF DESCRIPTION OF THE DRAWINGS

FIG. 1 depicts antioxidant capacities of extracts of Terminalia chebula including a TC/enriched tannoid blend in accordance with an embodiment of the present invention in an ABTS radical cation decolorisation assay. Trolox equivalent antioxidant capacity (TEAC), as used in the present disclosure, is expressed in μmoles of trolox equivalents/gm sample.

FIG. 2 depicts antioxidant capacities of extracts of Terminalia chebula including a TC/enriched tannoid blend in accordance with an embodiment of the present invention in a DPPH radical decolorisation assay. Results are expressed as IC₅₀ values (μg/ml) of samples, since trolox does not show sensitivity in this assay. Lower IC₅₀ values are indicative of higher antioxidant capacity.

FIG. 3 depicts antioxidant capacities of extracts of Terminalia chebula including a TC/enriched tannoid blend in accordance with an embodiment of the present invention in a Ferric reducing antioxidant potential assay (FRAP).

FIG. 4 depicts antioxidant capacities of extracts of Terminalia chebula including a TC/enriched tannoid blend in accordance with an embodiment of the present invention in a hydroxyl radical scavenging assay by 2-deoxyribose degradation method.

FIG. 5 depicts antioxidant capacities of extracts of Terminalia chebula including a TC/enriched tannoid blend in accordance with an embodiment of the present invention in a superoxide radical scavenging assay in NADH-NBT-PMS method. Results are expressed as IC₅₀ values (μg/ml) of samples, since trolox does not show sensitivity in this assay. Lower IC₅₀ values are indicative of higher antioxidant capacity.

FIG. 6 depicts antioxidant capacities of extracts of Terminalia chebula including a TC/enriched tannoid blend in accordance with an embodiment of the present invention in a hydrogen peroxide scavenging assay.

FIG. 7 depicts total phenolic content of the extracts of Terminalia chebula including a TC/enriched tannoid blend in accordance with an embodiment of the present invention determined by Folin-Ciocalteu method. Results are expressed in μmoles of gallic acid equivalents/gm sample.

FIG. 8 depicts antioxidant capacities of extracts of Terminalia chebula including a TC/enriched tannoid blend in accordance with an embodiment of the present invention in a nitric oxide radical scavenging assay.

FIG. 9 depicts antioxidant capacities of extracts of Terminalia chebula including a TC/enriched tannoid blend in accordance with an embodiment of the present invention in a peroxynitrite (ONOO⁻) scavenging assay. Results are expressed in μmoles of ascorbic acid equivalents/gm sample.

FIG. 10 depicts antioxidant capacities of extracts of Terminalia chebula including a TC/enriched tannoid blend in accordance with an embodiment of the present invention in an assay for inhibition of erythrocyte membrane lipid peroxidation.

FIG. 11 depicts antioxidant capacities of extracts of Terminalia chebula including a TC/enriched tannoid blend in accordance with an embodiment of the present invention in an assay for inhibition of lipid peroxidation in goat brain homogenate.

### DETAILED DESCRIPTION

In an embodiment, a Terminalia chebula extract containing a hydrolyzable tannoid blend is provided. A method for extracting Terminalia chebula to obtain an enriched hydrolyzable tannoid powder is provided.

Studies cited above used extracts of T. chebula whole fruit. However, T. chebula contains several bioactive components, including chebulagic acid, chebulinic acid, chebulic acid and other low molecular weight hydrolyzable tannoids (LMwHTs). Many studies, some of which are described below, have also been done on the individual bioactives of T. chebula.

Tannins may be divided into two groups: (a) hydrolyzable tannoids (HTs), which are esters of a polyol or sugar, usually glucose, with one or more trihydroxybenzenecarboxylic acids (i.e., gallates), and (b) derivatives of procyanidins, flavanols or flavanones, so-called condensed tannins HTs are molecules with a polyol (polyfunctional alcohols, generally D-glucose or its derivatives and phenols, namely galloyl and ellagoyl moieties) as a central core. The hydroxyl groups of these carbohydrates are partially or totally esterified with phenolic carboxylic acids like gallic acid (gallotannins), ellagic acid (ellagitannins) or both (gallo-ellagitannins)

Chebulagic acid, depicted in the compound of formula (1), is a tannoid (low Mw polyphenolic) member of the tannin family and has been found as a constituent in many medicinal plants. Chebulagic acid is chemically named as beta-1-O-galloyl-2,4-chebuloyl-3,6-(R)-hexahydroxydiphenoyl-D-glucose.

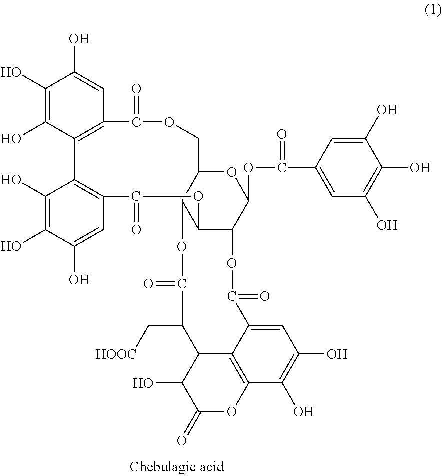 [SMILES: O=C(O)CC1C(=O)OC2C3COC(=O)c4cc(O)c(O)c(O)c4-c4c(cc(O)c(O)c4O)C(=O)OC2C(OC(=O)c2cc(O)c(O)c4c2C1C(O)C(=O)O4)C(OC(=O)c1cc(O)c(O)c(O)c1)O3]

Hydrolyzable tannoids have been reported as key bioactive components of T. chebula. Chebulagic acid and chebulinic acid are the two major bioactive hydrolyzable tannoids of T. chebula. Chebulagic acid (“CA”), a natural antioxidant, has shown potent anti-inflammatory effects in LPS-stimulated RAW 264.7, a mouse macrophage cell line. These effects were exerted via inhibition of NO and PGE₂ production and down-regulation of iNOS, COX-2,5-LOX, TNF-α and IL-6. CA inhibited NF-κB activation by LPS, and this was associated with the abrogation of IKB-α phosphorylation and subsequent decreases in nuclear p50 and p65 protein levels (D. B. Reddy, et al., Biochemical and Biophysical Research Communications. (2009) 381: 112-117).

Chebulagic acid has shown potent COX-LOX dual inhibition activity with IC₅₀ values of 15±0.288, 0.92±0.011 and 2.1±0.057 μM for COX-1, COX-2 and 5-LOX, respectively. CA also exhibited anti-proliferative activity against HCT-15, COLO-205, MDA-MB-231, DU-145 and K562 cell lines. Further mechanistic studies on COLO-205 cells revealed induction of apoptosis by chebulagic acid (D. B. Reddy, et al., J. Ethnopharmacol. (2009) 124: 506-12).

Chebulagic acid, isolated from Terminalia chebula Retz, proved to be a reversible and non-competitive inhibitor of maltase with a K(i) value of 6.6 μM. The inhibitory influence of chebulagic acid on the maltase-glucoamylase complex was more potent than on the sucrase-isomaltase complex. The magnitude of alpha-glucosidase inhibition by chebulagic acid was greatly affected by its origin. These results show a use for chebulagic acid in managing type-2 diabetes (Y. N. Huang et al., Biosci. Biotechnol. Biochem. (2008) 72: 601-3).

Chebulagic acid has also been shown to synergize the cytotoxicity of doxorubicin in human hepatocellular carcinoma through COX-2 dependent modulation of MDR-1. Chebulagic acid increased the accumulation of doxorubicin in a concentration dependant manner and also enhanced the cytotoxicity of doxorubicin in HepG2 cells by 20 fold. Quantitation of interaction by calculating Combination Index (CI) showed a strong synergistic interaction between chebulagic acid and doxorubicin in terms of cell growth inhibition (C. Achari, et al., Med. Chem. (2011) 7: 432-42).

Herpes simplex virus 1 (HSV-1) is a common human pathogen that causes lifelong latent infection of sensory neurons. Non-nucleoside inhibitors that can limit HSV-1 recurrence are particularly useful in treating immunocompromised individuals or cases of emerging acyclovir-resistant strains of herpes virus. Chebulagic acid and punicalagin, two hydrolyzable tannoids isolated from the dried fruits of Terminalia chebula Retz. (Combretaceae), have been found to inhibit HSV-1 entry at noncytotoxic doses in A549 human lung cells by blocking the interactions between cell surface glycosaminoglycans and HSV-1 glycoproteins (L. T. Lin, et al., J. Virol. (2011) 85: 4386-98).

Chebulagic acid has been reported to suppress the onset and progression of collagen-induced arthritis in mice through immune suppression (anticollagen IgG, IL-10, IL-6) via the induction of TGFbeta and CD4+, CD25+ T cells (P.M. Lee, S. I. Hyun, et al., Arthritis Rheum. (2005) 52: 345-53).

Chebulagic acid has been reported to possess cytotoxic properties against PRMI-7951 melanoma cells (Y. Kashiwada, et al., J. Nat. Prod. (1992) 55: 1033-43).

Chebulinic acid, depicted in the compound of formula (2), is another tannoid member of the tannin family derived from galloyl glucose. Chebulinic acid is chemically named as 1,3,6-tri-O-galloyl-2,4-chebuloyl-beta-D-glucose.

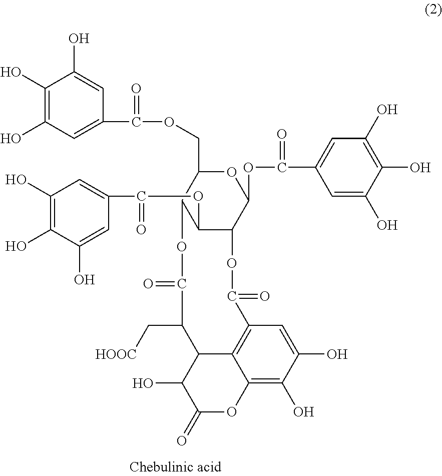 [SMILES: O=C(O)CC1C(=O)OC2C(COC(=O)c3cc(O)c(O)c(O)c3)OC(OC(=O)c3cc(O)c(O)c(O)c3)C(OC(=O)c3cc(O)c(O)c4c3C1C(O)C(=O)O4)C2OC(=O)c1cc(O)c(O)c(O)c1]

Chebulinic acid and Tellimagrandin I have been shown to exert anti-tumor properties in human cervical carcinoma HeLa cells (Z. C. Yi, et al., Cancer Lett. (2006) 242: 77-87).

Chebulinic acid inhibited the hemoglobin synthesis of butyric acid and hemin-treated K562 cells in a concentration-dependent manner. Chebulinic acid has also been reported to inhibit the erythroid differentiation likely through changing transcriptional activation of differentiation relative genes (Z. C. Yi, et al., Acta Pharmacol Sin. (2004) 25: 231-8).

Chebulinic acid, tannic acid and ellagic acid were reported to be the growth inhibitory phenolics of T. chebula fruits against malignant cell lines including a human (MCF-7) and mouse (S115) breast cancer cell line, a human osteosarcoma cell line (HOS-1), a human prostate cancer cell line (PC-3) and a non-tumorigenic, immortalized human prostate cell line (PNT1A) (A. Saleem, et al., J Ethnopharmacol. (2002) 81: 327-36).

Chebulinic acid has been shown to elicit blood pressure lowering effect in rats, likely mediated via the decrease in cardiac output resulting from reduced left ventricular contraction (Y. Y. Guan, et al., Clin Exp Pharmacol Physiol. (1996) 23: 747-50).

Chebulinic acid and punicalin were able to block the binding of HIV rgp120 to CD4. These compounds were not toxic to stimulated human peripheral blood lymphocytes at concentrations ten times above their maximal effective concentration (J. L. Weaver, et al., Biochem. Pharmacol. (1992) 43: 2479-80).

Gallic acid (GA) and chebulic acid (CA) were isolated from the extract of the herbal medicine Kashi (myrobalan, the fruit of T. chebula) as active principle that blocked the cytotoxic T-lymphocyte (CTL)-mediated cytotoxicity. Granule exocytosis in response to anti-CD3 stimulation was also blocked by GA and CA at the equivalent concentrations (S-I. Hamada, et al., Biological & Pharmaceutical Bulletin. (1997) 20: 1017-1019).

Chebulagic acid and chebulinic acid have been shown to possess antifibrotic activity through the inhibition of the Smad pathway (H. Y. Chuang, et al., J. Sci. Food Agric. (2011) 91:2777-84). Chebulagic acid, chebulinic acid, and other phenolics isolated from T. chebula, showed stronger DPPH radical scavenging and melanin inhibitory activities than ascorbic acid, butylated hydroxyl toluene, alpha-tocopherol, arbutin and kojic acid (A. Manosroi, et al., Nat. Prod. Res. (2010) 24: 1915-26).

As evidenced by the extensive and significant pharmacological activity of the bioactive constituents and/or components of T. chebula, there is a need for these bioactives to be obtained at high levels, up to the maximum possible extent in an extract of this plant. In one embodiment, the present invention contemplates a T. chebula extract including an enriched hydrolyzable tannoid blend. The enriched hydrolyzable tannoid blend can include bioactive hydrolyzable tannoids selected from chebulagic acid, chebulinic acid, other low molecular weight hydrolyzable tannoids, and combinations thereof.

Several samples of T. chebula extract available in the market from different suppliers were obtained and quantitative analysis of the bioactives was performed using high pressure liquid chromatography (HPLC), the results of which are presented in Table 1.

HPLC Analytical Method

Each sample is derived from a commercially available standardized extract of Terminalia chebula fruit for nutraceutical and cosmetic use. The active constituents include a combination of chebulagic acid, chebulinic acid, and other Low Molecular weight Hydrolysable Tannoids (LMwHTs).

Sample Preparation. 50 mg of T. chebula powdered extract (aqueous extract) is dispersed in 10 ml of double distilled water. The dispersion is sonicated for 10 minutes and then centrifuged at 8500 rpm for 10 minutes. The resulting supernatant at a concentration of 5 mg/ml is injected (20 μl) for a typical HPLC run cycle.

HPLC Conditions.

Column: reversed phase C18 LiChroCART, 250 mm 1×4 mm i.d., 5 μm particle diameter. (E. Merck, Germany).

Column temp.: ambient.

Eluant: aqueous phase [A]: 0.1% formic acid; organic phase [B]acetonitrile (ACN).

Flow rate: 0.8 ml/min.

Run Time: 46 min. Gradient: B 0-15% (1 min), 15-25% (35 min), 25-60% (9 min) and re-equilibriation 60-0% (1 min).

UV detection at 270 nm; Waters HPLC Model 515 with PDA detector (Waters™ 2996, Photodiode Array Detector), evaluation with Empower.

HPLC Evaluation Method. The method was developed with external standards and evaluation of area of peaks using respective calibration equation.

A. Preparation of Linear Regression Equation of Chebulinic Acid.

A reference standard of chebulinic acid (98% w/w pure, isolated from T. chebula fruits using Sephadex G-50 (Amersham Bioscience) and Low Pressure Chromatography (Bio-Rad)) was dissolved in distilled water to prepare three different concentrations (40 μg/20 μl, 20 μg/20 μl and 10 μg/20 μl) required for preparation of calibration curve. The prepared concentrations were subjected to HPLC analysis. The peak areas were calculated for each dilution, and the respective concentration was plotted against the peak area. The amount of chebulinic acid, chebulagic acid, chebulinic acid equivalents and chebulagic acid equivalents were determined using regression equation of the calibration curve obtained as follows Y=209490383x+3958610 with a correlation coefficient of 1.0. Y is the peak area and X is the concentration in μg/20 μl.

B. Calculation Formulae

1. Chebulagic acid: The area of the peak appearing at t_{R} 13.74 minutes is considered as Chebulagic acid and the amount calculated using the above mentioned calibration equation of Chebulinic acid (Y=209490383x+3958610) and the formula as follows. Chebulagic acid present in the extract (% w/w)=[Amount of chebulagic acid obtained using calibration equation (μg)/Amount of extract injected (μg)]×100.

2. Chebulinic acid: The area of the peak appearing at t_{R} 20.66 minutes is considered as Chebulinic acid and the amount calculated using the above mentioned calibration equation of Chebulinic acid (Y=209490383x+3958610) and the formula as follows. Chebulinic acid present in the extract (% w/w)=[Amount of chebulinic acid obtained using calibration equation (μ)/Amount of extract injected (μg)]×100.

3. Other LMwHTs: The sum of the area of peaks appearing between 7-13 minutes, 15.681 minutes, 19.88, 23.107 minutes are added and the amount of other LMwHTs calculated using the calibration equation of Chebulinic acid (Y=209490383x+3958610) and the formula as follows. Other LMwHTs present in the extract (% w/w)=[Combined amount of Other LMwHTs obtained using calibration equation (μg)/Amount of extract injected (μg)]×100.

Comparative HPLC

TABLE 1

| | Market | Market | Market |
|---|---|---|---|
| | Sample 1 | Sample 2 | Sample 3 |
| Bioactive | % w/w | % w/w | % w/w |
| Chebulagic acid | 0.49% | 0.03% | 0.70% |
| Chebulinic acid | 0.25% | 1.24% | 0.91% |
| Other LMWtHTs* | 19.67 | 40.96 | 16.18 |

*LMwHTs = Low molecular weight hydrolysable tannoids.

As shown from the results in Table 1 above, T. chebula extracts currently available in the market are very low in most of the bioactives. Thus, there is a need for T. chebula extracts in which the contents of bioactives are much higher—collectively to the extent of about 50%, or even greater than 50%. There is also a need to develop improved extraction conditions for enriching the extract with several different bioactives, including but not limited to chebulagic acid, chebulinic acid and other low molecular weight hydrolyzable tannoids (e.g., galloyl glucose precursors of tannoids), the presence of which is indicated in some better quality fruits when analyzed by HPLC. It is also important, from environmental and health points of view, to have a method of extraction which is completely aqueous. All aqueous extraction can minimize exposure to and ingestion of potentially dangerous or toxic solvents by an individual human or animal subject.

Several methods were tried to accomplish the objective of obtaining a T. chebula extract with enriched bioactives. In an embodiment, a process of making a T. chebula extract containing a hydrolyzable tannoid blend is provided. The invention further relates to a method for extracting Terminalia chebula to obtain an enriched hydrolyzable tannoid powder.

Herbal extracts can be made by grinding the herbs into a fine powder and suspending the powder into a solution of alcohol, water, and mixtures thereof. The suspension is regularly agitated or pulverized (e.g., by ultrasonication) over time and then pressed through a filtering medium to extract the bio-active ingredients.

In an embodiment, the extraction process of the current invention includes the steps of: providing fruits of T. chebula; pulverizing or grinding the T. chebula to a powder; extracting the T. chebula powder with an extraction solvent or solvent mixture, optionally, with heating, to provide a T. chebula enriched extract; and concentrating or drying the T. chebula enriched extract to provide a hydrolyzable tannoid enriched T. chebula powder. Aqueous solvent is preferred. A particularly preferred solvent is water. Useful extraction temperatures can range from about 25° C. (ambient) to about 90° C. Particularly useful extraction temperatures can range from about 25° C. to about 80° C.

Useful extraction times in conjunction with maintaining the useful temperatures can range from about 2 hours to about 16 hours. A particularly useful extraction time range at about 25° C. is from about 12 hours to about 16 hours. Length and temperature of extraction may be varied at atmospheric pressure (i.e., approx. 1 atm). It is contemplated that pressure can be varied in the extraction process, for example, by use of a commercial pressure reactor apparatus.

The extraction process can also include drying the extracted sample. Suitable drying methods include spray drying, lyophilization, freeze drying, vacuum drying (with or without heating), evaporation (with or without heating), and concentration under vacuum. Once isolated or obtained the hydrolyzable tannoid enriched T. chebula extract powder may be processed by any suitable means, including grinding, milling, sieving, sizing, and the like. The obtained hydrolyzable tannoid enriched T. chebula extract powder may be prepared in any suitable particle size or particle size range.

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. In accordance with one embodiment, suitable nutraceutically acceptable carriers can include ethanol, aqueous ethanol mixtures, water, fruit and/or vegetable juices, and combinations thereof. Similarly, the compositions as described may be used for pharmaceutical compositions, cosmetic compositions, or skin care compositions, and may be administered in combination with a pharmaceutically or cosmeceutically acceptable carrier, as appropriate.

Solid nutritional compositions for oral administration may optionally contain, in addition to the above enumerated nutritional composition ingredients or compounds: carrier materials such as corn starch, gelatin, acacia, microcrystalline cellulose, kaolin, dicalcium phosphate, calcium carbonate, sodium chloride, alginic acid, and the like; disintegrators including, microcrystalline cellulose, alginic acid, and the like; binders including acacia, methylcellulose, sodium carboxymethylcellulose, polyvinylpyrrolidone, hydroxypropyl methylcellulose, ethyl cellulose, and the like; and lubricants such as magnesium stearates, stearic acid, silicone fluid, talc, waxes, oils, colloidal silica, and the like. The usefulness of such excipients is well known in the art.

In a preferred embodiment, the nutritional composition may be in the form of a liquid. In accordance with this embodiment, a method of making a liquid composition is provided.

Liquid nutritional compositions for oral administration in connection with a method for preventing and/or treating free radical-induced illnesses or ailments, or various other inflammatory conditions or ailments, can be prepared in water or other aqueous vehicles. In addition to the above enumerated ingredients or compounds, liquid nutritional compositions can include suspending agents such as, for example, methylcellulose, alginates, tragacanth, pectin, kelgin, carrageenan, acacia, polyvinylpyrrolidone, polyvinyl alcohol, and the like. The liquid nutritional compositions can be in the form of a solution, emulsion, syrup, gel, or elixir including or containing, together with the above enumerated ingredients or compounds, wetting agents, sweeteners, and coloring and flavoring agents. Various liquid and powder nutritional compositions can be prepared by conventional methods. Various ready-to-drink formulations (RTD's) are contemplated.

The methods described above may be further understood in connection with the following Examples. The results of an extraction process depend upon the solvent used, temperature of extraction and duration of the extraction process. In several embodiment of this invention, these factors can be optimized to isolate and/or enrich and preserve the bioactives of T. chebula. T. chebula as used in the following examples was obtained from Ramakrishna Mission Ashrama, Narendrapur (Kolkata, West Bengal, India).

### Example 1A

The effect of temperature and duration of extraction were first optimized using water as an extraction solvent. In a typical experiment, dried whole fruit and fruit pericarp portions of T. chebula (50 g) were pulverized and the resulting powder was extracted separately with distilled water (300 ml) for 16 hours at room temperature, kept without stirring. The samples were then spray dried and analyzed for bioactives (Chebulagic acid, Chebulinic acid and other LMwHTs) by HPLC (as discussed above). The results are incorporated in Table 2.

Effect of aqueous extraction, at room temperature for 16 hrs, on the bioactives contents of T. chebula.

TABLE 2

| | Aqueous extract of | Aqueous extract of |
|---|---|---|
| | T chebula | T chebula |
| Bioactives | whole fruit powder | pericarp powder |
| Chebulinic acid (% w/w) | 17.76 | 24.36 |
| Chebulagic acid (% w/w) | 3.03 | 14.21 |
| Other LMwHTs (% w/w) | 21.39 | 20.10 |
| Yield of Extractive (% w/w) | 24.52 | 51.80 |

The results indicated that the whole fruit extract and fruit pericarp extract yielded better results, in comparison to the marketed extract samples (cf. Table 1), with respect to their chebulagic acid and chebulinic acid contents. Also, it was noted that fruit pericarp extract contained higher amounts of chebulagic acid and chebulinic acid than whole fruit extract. Due to the higher contents of chebulagic acid and chebulinic acid in the fruit pericarp, this part was selected for further experiments.

### Example 1B

In another experiment, fruit pericarp portions of T. Chebula (50 g) were pulverized and the resulting powder was extracted with distilled water (300 ml) for 12 hours, with a continuous stirring (400 RPM), separately at different temperatures 30±5° C., 40±5° C., 60±5° C. and 80±5° C. using a pressure reactor. Aliquots of the samples were withdrawn at different time intervals during extraction, spray dried and analyzed for bioactives (Chebulagic acid, Chebulinic acid and other LMwHTs) by HPLC (as discussed above). The results are incorporated in Tables 3-6. As used herein, “0 hour” extraction refers to “instant extraction,” which for the purpose of this disclosure means distilled water was added to the pulverized powder on a thermostatic water bath, stirred for 5 minutes and an aliquot of the sample withdrawn and analyzed.

Effect of Duration of Aqueous Extraction at 30±5° C., on the Bioactives Content of T. chebula.

TABLE 3

| | 0 | 1 | 2 | 3 | 4 | 6 | 8 | 12 |
|---|---|---|---|---|---|---|---|---|
| Bioactives | Hr* | Hr | Hr | Hr | Hr | Hr | Hr | Hr |
| Chebulinic | 17.11 | 21.91 | 22.46 | 23.40 | 20.35 | 22.13 | 15.89 | 13.45 |
| acid | | | | | | | | |
| (% w/w) | | | | | | | | |
| Chebulagic | 8.15 | 13.54 | 13.13 | 14.97 | 17.13 | 17.56 | 20.46 | 15.20 |
| acid | | | | | | | | |
| (% w/w) | | | | | | | | |
| Other | 4.55 | 16.46 | 8.66 | 11.01 | 12.04 | 9.89 | 14.91 | 10.53 |
| LMwHTs | | | | | | | | |
| (% w/w) | | | | | | | | |
| Yield of | 17.70 | 36.90 | 38.52 | 39.42 | 36.30 | 37.62 | 30.18 | 33.54 |
| extractive | | | | | | | | |
| (% w/w) | | | | | | | | |

*indicates instant extraction

Effect of Duration of Aqueous Extraction at 40±5° C., on the Bioactives Content of T. chebula.

TABLE 4

| | 0 | 1 | 2 | 3 | 4 | 6 | 8 | 12 |
|---|---|---|---|---|---|---|---|---|
| Bioactives | Hr | Hr | Hr | Hr | Hr | Hr | Hr | Hr |
| Chebulinic | 17.11 | 24.73 | 25.54 | 26.40 | 23.73 | 23.51 | 20.05 | 12.22 |
| acid | | | | | | | | |
| (% w/w) | | | | | | | | |
| Chebulagic | 8.15 | 15.68 | 15.20 | 16.93 | 14.04 | 15.52 | 14.93 | 12.90 |
| acid | | | | | | | | |
| (% w/w) | | | | | | | | |
| Other | 4.55 | 6.24 | 9.15 | 9.10 | 5.67 | 11.37 | 11.48 | 9.62 |
| LMwHTs | | | | | | | | |
| (% w/w) | | | | | | | | |
| Extractive | 17.70 | 48.54 | 44.58 | 47.82 | 48.14 | 45.90 | 44.10 | 45.90 |
| value | | | | | | | | |
| (% w/w) | | | | | | | | |

* indicates instant extraction

Effect of Duration of Aqueous Extraction at 60±5° C., on the Bioactives Content of T. chebula.

TABLE 5

| | 0 | 1 | 2 | 3 | 4 | 6 | 8 | 12 |
|---|---|---|---|---|---|---|---|---|
| Bioactives | Hr* | Hr | Hr | Hr | Hr | Hr | Hr | Hr |
| Chebulinic | 17.11 | 20.68 | 22.48 | 20.11 | 21.21 | 20.46 | 17.37 | 10.53 |
| acid | | | | | | | | |
| (% w/w) | | | | | | | | |
| Chebulagic | 8.15 | 14.08 | 14.05 | 12.38 | 12.40 | 13.99 | 12.09 | 9.00 |
| acid | | | | | | | | |
| (% w/w) | | | | | | | | |
| Other | 4.55 | 9.87 | 8.92 | 8.26 | 8.48 | 9.73 | 10.31 | 5.98 |
| LMwHTs | | | | | | | | |
| (% w/w) | | | | | | | | |
| Yield of | 17.70 | 45.78 | 47.34 | 47.64 | 49.38 | 50.28 | 45.48 | 45.90 |
| extractive | | | | | | | | |
| (% w/w) | | | | | | | | |

*indicates instant extraction

Effect of Duration of Aqueous Extraction, at 80±5° C., on the Bioactives Contents of T. chebula.

TABLE 6

| | 0 | 1 | 2 | 3 | 4 | 6 | 8 | 12 |
|---|---|---|---|---|---|---|---|---|
| Bioactives | Hr* | Hr | Hr | Hr | Hr | Hr | Hr | Hr |
| Chebulinic | 22.46 | 12.76 | 12.89 | 12.08 | 9.21 | 6.40 | 5.13 | 1.01 |
| acid | | | | | | | | |
| (% w/w) | | | | | | | | |
| Chebulagic | 14.14 | 8.02 | 7.89 | 7.58 | 7.05 | 4.84 | 1.52 | −0.61 |
| acid | | | | | | | | |
| (% w/w) | | | | | | | | |
| Other | 18.06 | 15.75 | 24.27 | 45.76 | 43.65 | 40.94 | 50.96 | 40.82 |
| LMwHTs | | | | | | | | |
| (% w/w) | | | | | | | | |
| Yield of | 45 | 63.28 | 58.24 | 53.76 | 51.92 | 40.32 | 38.50 | 36.09 |
| Extractive | | | | | | | | |
| (% w/w) | | | | | | | | |

*indicates instant extraction

As shown above, chebulagic acid and chebulinic acid were extracted more effectively and more efficiently in distilled water at room temperature for 16 hrs than at higher temperatures. Likewise, extraction at 40±5° C. for 3 hrs also resulted in better chebulagic acid and chebulinic acid contents than extraction at other temperatures, e.g. 30±5° C., 60±5° C. and 80±5° C. Extraction results at 80±5° C., at different time points, indicated that chebulagic acid and chebulinic acid are thermolabile and transformed into other compounds with increasing temperature and time. These findings suggest that for a composition having maximum chebulagic acid, chebulinic acid and LMwHTs, the optimum extraction conditions would be extraction at room temperature for 16 hours or extraction at 40±5° C. for about 3 hrs. However, it should be noted that the time of extraction may be substantially lowered by decreasing the particle size of the dried raw material, using stirring, performing the extraction process at higher pressures than the atmospheric pressure, altering the ratio of the fruit powder and the extraction solvent, and combinations of these parameters, or variation of other physical processing parameters.

Thus, the above extraction procedures yielded an enriched hydrolyzable tannoid blend. It is exemplary that other temperatures may be useful for all-aqueous extraction, such as, for example, 30° C., 40° C., 50° C., 60° C., 70° C., and 80° C. Useful extraction times may range from about 0 hours to about 24 hours. Other suitable extraction times may range from about 0 hours to about 6 hours, or from about 0 hours to about 4 hours, or from about 0 hours to about 3 hours, or from about 0 hours to about 2 hours.

Additionally, the above extraction procedures yielded total extractive tannin or tannoid compositions, that is, T. chebula/enriched tannoids of about 45% by weight, or greater, based on the total weight of the extract composition. In one embodiment, the total yield TC/enriched tannoids is about 50% by weight, or greater. In these embodiments, chebulinic acid and chebulagic acid can make up the greatest proportion of the total tannoids.

### Example 2

The effect of different extract drying procedures (freeze drying, spray drying, vacuum drying) on the content of bioactives was determined. Dry pulp of Terminalia chebula was first pulverized and blended in a mini-blender. The resulting mixture was passed through 22 mesh sieve to get uniform particle size of powder. 50 μm powder was then extracted with 300 ml water (1:6 w/v, solid-to-solvent ratio) at 40° C. for 3 hrs in a pressure reactor. The mixture was continuously stirred at a speed of 400 rpm. After completion of extraction, total sample was withdrawn, centrifuged for 5 minutes at 8000 rpm and filtered through filter paper.

Total filtrate (water extract) was divided in four parts and dried by different methods as follows:

1) 20 ml extract was lyophilized.

2) 20 ml extract was taken on a petri dish and kept on a steam bath for 1 hr.

3) 20 ml extract was concentrated on rotary evaporator under reduced pressure and kept overnight in a vacuum dryer.

4) Remaining extract was spray dried.

The dried extracts were analyzed for bioactives (Chebulagic acid, chebulinic acid and other LMwHTs) by HPLC (as discussed above). The results are incorporated in Table 7.

Effect of Different Drying Conditions on the Bioactives (Chebulagic Acid, Chebulinic Acid and Other LMwHTs) Content of T. chebula Extract.

TABLE 7

| | | Spray | Vacuum | Extract |
|---|---|---|---|---|
| | Lyophilized | dried | dried | dried on |
| Bioactives | extract | extract | extract | water bath |
| Chebulinic acid (% w/w) | 26.76 | 24.56 | 21.33 | 18.11 |
| Chebulagic acid (% w/w) | 15.83 | 15.85 | 14.61 | 11.96 |
| Other LMWHTs (% w/w) | 9.49 | 8.62 | 9.02 | 11.8 |

The above results as shown in Table 7 indicate that freeze drying (i.e., lyophilization) and spray drying yielded better chebulagic acid and chebulinic acid contents in the extract than vacuum drying.

### Example 3

The effect of different solvent extractions on the bioactives content was determined at 80±5° C. and up to 6 hrs of extraction. In a typical experiment, dried fruit pericarp portions of the T. chebula were pulverized and extracted separately with one of the following solvents: 40:60 (v/v) methanol:water; 75:25 (v/v) methanol:water; 25:75 (v/v) methanol:water. All extractions were carried out with a fruit powder:solvent ratio of 1:6 wt/vol (i.e., 50 g T. chebula fruit powder extracted with 300 ml solvent). All extractions were conducted for 6 hours at 80±5° C. using a thermostatic water bath. Aliquots of the samples were withdrawn at different time intervals during extraction, spray dried and analyzed for bioactives (Chebulagic acid, Chebulinic acid and other LMwHTs) by HPLC (as above). The results are incorporated in Tables 8-10.

Effect of Duration of Methanol:Water (40:60 v/v) Extraction, at 80±5° C., on the Bioactives Contents of T. chebula.

TABLE 8

| | 0 | 1 | 2 | 3 | 4 | 5 | 6 |
|---|---|---|---|---|---|---|---|
| Bioactives | Hr* | Hr | Hr | Hr | Hr | Hr | Hr |
| Chebulinic | 20.87 | 20.04 | 19.20 | 14.78 | 12.92 | 10.35 | 12.87 |
| acid | | | | | | | |
| (% w/w) | | | | | | | |
| Chebulagic | 12.12 | 12.71 | 12.50 | 10.34 | 7.83 | 7.40 | 9.32 |
| acid | | | | | | | |
| (% w/w) | | | | | | | |
| Other | 12.91 | 18.61 | 62.15 | 35.62 | 35.93 | 40.81 | 40.30 |
| LMwHTs | | | | | | | |
| (% w/w) | | | | | | | |
| Yield of | 51.33 | 68.60 | 67.20 | 62.04 | 62.87 | 67.80 | 65.71 |
| Extractive | | | | | | | |
| (% w/w) | | | | | | | |

*indicates instant extraction

Effect of Duration of Methanol:Water (75:25 v/v) Extraction, at 80±5° C., on the Bioactives Contents of T. chebula.

TABLE 9

| | 0 | 1 | 2 | 3 | 4 | 5 | 6 |
|---|---|---|---|---|---|---|---|
| Bioactives | Hr* | Hr | Hr | Hr | Hr | Hr | Hr |
| Chebulinic | 22.88 | 21.03 | 20.59 | 9.26 | 15.08 | 14.41 | 13.67 |
| acid | | | | | | | |
| (% w/w) | | | | | | | |
| Chebulagic | 11.78 | 13.42 | 10.98 | 8.25 | 7.20 | 4.39 | 5.77 |
| acid | | | | | | | |
| (% w/w) | | | | | | | |
| Other | 15.67 | 19.03 | 24.59 | 27.29 | 32.81 | 20.11 | 31.06 |
| LMwHTs | | | | | | | |
| (% w/w) | | | | | | | |
| Yield of | 49.00 | 69.91 | 70.08 | 63.61 | 70.84 | 64.80 | 70.69 |
| Extractive | | | | | | | |
| (% w/w) | | | | | | | |

*indicates instant extraction

Effect of Duration of Methanol:Water (25:75 v/v) Extraction, at 80±5° C., on the Bioactives Contents of T. chebula.

TABLE 10

| | 0 | 1 | 2 | 3 | 4 | 5 | 6 |
|---|---|---|---|---|---|---|---|
| Bioactives | Hr* | Hr | Hr | Hr | Hr | Hr | Hr |
| Chebulinic | 22.70 | 22.25 | 16.77 | 18.62 | 9.70 | 6.82 | 6.69 |
| acid | | | | | | | |
| (% w/w) | | | | | | | |
| Chebulagic | 12.64 | 8.00 | 9.58 | 8.02 | 5.27 | 5.74 | 3.51 |
| acid | | | | | | | |
| (% w/w) | | | | | | | |
| Other | 26.30 | 30.03 | 40.03 | 25.47 | 47.93 | 43.55 | 42.08 |
| LMwHTs | | | | | | | |
| (% w/w) | | | | | | | |
| Yield of | 56.00 | 65.99 | 65.28 | 69.25 | 65.32 | 63.30 | 64.83 |
| Extractive | | | | | | | |
| (% w/w) | | | | | | | |

*indicates instant extraction

The results as shown in Tables 8-10 indicated that instant extraction up to about 1 hour extraction of dried pericarp of T. chebula with different ratios of methanol:water yielded high levels of chebulinic acid (about 20-23%), and chebulagic acid (about 8-14%). However, it was observed that with increasing extraction time at 80±5° C., the contents of chebulinic acid and chebulagic acid were generally found to be decreased, suggesting their susceptibility to degradation at the higher temperature.

In conclusion, it was observed that chebulinic acid and chebulagic acid were highly susceptible to thermal degradation under certain extraction conditions. Notably, chebulinic acid and chebulagic acid (but not necessarily other low molecular weight hydrolyzable tannoids) were substantially decreased with increasing temperature and increasing exposure time. Without being bound by any theory, it is apparent that due to thermal instability, these compounds were found at low or very low levels in market samples of T. chebula extracts (which are known to be prepared by conventional hot solvent extraction and conventional drying processes). The above examples show that extraction and drying conditions and steps were optimized in order to preserve high levels of chebulinic acid and chebulagic acid, thereby increasing their content levels in the final T. chebula extract compositions.

In another embodiment, it is expected that a combination of the above extractive procedures may be carried out, without limitation, in a variety of multiple or sequential extraction steps, in order to obtain other optimized levels of the desired bioactive components. In particular, it is expected that by using the techniques applied above, optimized levels, or higher levels, of any one or all of the bioactive components, can be achieved.

In view of the examples, it is expected that use of a hydrolyzable tannoid enriched T. chebula extract made in accordance with the principles of the invention, in a pharmaceutical or nutraceutical composition, would possess improved properties for the treatment or prevention of diseases related to inflammation, inflammatory processes, oxidative stress, or free radicals.

### Example 4

Comparative Antioxidant Activity of T. chebula Extracts in In Vitro Models.

Four different T. chebula based extract powders were tested, including (a) the enriched hydrolyzable tannoid blend of Example 1A (Table 2, derived from fruit pericarp), and (b) Market Samples 1-3 (Table 1, as discussed above). Each sample was a free-flowing powder.

Background Regarding Free Radicals and Antioxidants.

Free radicals are atoms or groups of atoms containing at least one unpaired electron in their orbitals, and, in one exemplary route, can be formed when oxygen interacts with certain molecules. Once formed in abundance (or when having a high localized concentration), these highly reactive species can start a chain reaction, causing damage to many biomolecules. Their main danger comes from the damage they can do when they react with important cellular components such as DNA, mitochondria, cellular proteins or the lipids present in the cell membrane (S. F. Pala, et al., Adv. Mol. Biol. (2007) 1: 63-69). On the other hand, they play important role in different cellular processes, are involved in cytotoxicity, and as a defense against microorganisms and in neurotransmission, when present in low/moderate amounts (M. Valko, et al., Int. J. Biochem. Cell Biol. (2007) 39: 44-84). There are various types of free radicals that can be formed within the body. Reactive oxygen species (ROS) and reactive nitrogen species (RNS) are major components of this “free radical” system. The most deleterious ROS include hydroxyl radical, superoxide radical, singlet oxygen, and the like. The most deleterious RNS include nitric oxide and peroxynitrite anion. In addition to these two types of free radicals, atomic hydrogen, many heavy transition metals (e.g. iron, copper, zinc and manganese), halogenated compounds, many drugs, ionizing radiation, and environmental wastes (e.g. CO, asbestos, ozone, solvents etc.) also behave like sources of free radicals (G. S. Timmins and M. J. Davies, In: Electron Paramagnetic Resonance, The Royal Society of Chemistry, (Cambridge, UK), (1998) 1-49). The most common free radicals and/or oxidants present in the biological systems are described in Table 11 below.

General information about the most common ROS and RNS present in biological systems.

TABLE 11

| Free | | | |
|---|---|---|---|
| radical/ | Chemical | | |
| Oxidants | formula | Generating reaction | Damage caused |
| Hydroxyl | OH^{•} | Fe²⁺ + H₂O₂ → | Oxidation of |
| | | Fe³⁺ + OH^{•} + OH⁻ | almost all types |
| | | (Fenton reaction) | of biomolecules, |
| | | | especially DNA |
| Superoxide | O₂^{•-} | O₂ + e⁻ → O₂^{•-} | Oxidation of Fe—S |
| | | | cluster proteins |
| | | | to release Fe²⁺, |
| | | | responsible for |
| | | | generation of |
| | | | OH^{•} |
| Nitric | NO^{•} | Arginine → | Implicated in |
| oxide | | Citrulline + NO^{•} | Juvenile |
| | | | diabetes, |
| | | | multiple |
| | | | sclerosis, |
| | | | arthritis, and |
| | | | ulcerative colitis, |
| | | | when in excess |
| Peroxynitrite | ONOO⁻ | NO^{•} + O₂^{•-} → ONOO⁻ | Implicated in |
| anion | | | Heart disease, |
| | | | Alzheimer's |
| | | | disease, and |
| | | | atherosclerosis |
| Hydrogen | H₂O₂ | 2O₂^{•-} + 2H⁺ + 2e⁻ → | Acts as the |
| peroxide | | H₂O₂ + O₂ | source of OH^{•} |
| | | (Dismutation of | |
| | | superoxide radical) | |

Several enzyme systems within the body neutralize excess free radicals. One important component of the defense system that prevents the body from free radical damage includes antioxidants that can serve as chemical scavengers or quenchers of free radicals. Antioxidants are molecules which can safely interact with free radicals and terminate the chain reaction before vital molecules, or reactive sites, are damaged or chemically modified. Antioxidants are of two types: enzymatic antioxidants and non-enzymatic antioxidants. Enzymatic antioxidants include superoxide dismutase (SOD), glutathione peroxidase (GPx) and catalase (CAT). Non-enzymatic antioxidants include specific bioactive metabolites and several broad classes of agents, such as: ascorbic acid (Vitamin C), α-tocopherol (Vitamin E), glutathione (GSH), carotenoids, flavonoids, polyphenols, and molecules from other natural sources. Under normal physiological conditions, there is a balance between both the activities and the intracellular levels of these antioxidants within a living subject or organism. However, during stress, there may be an imbalance between the required beneficial, protective levels versus the actual physiological levels of these antioxidants in the subject. In these situations, antioxidants from one or more exogenous sources may be needed to overcome the effects of an assault by one or more types of free radicals. An ideal antioxidant should scavenge/neutralize different types of free radicals, e.g. OH., O₂.⁻, OOH., NO. and ONOO⁻ (or mixtures thereof), present in the biological systems.

Therefore, in embodiments of the present invention, a battery of in vitro antioxidant screens were selected to assess the comparative antioxidant activities of T. chebula samples, including TC/enriched tannoid blends prepared according to the principles of the present invention.

### Example 4A

ABTS radical cation decolorisation assay (N. Pellegrini, et al., J. Nutr. (2003) 133: 2812-2819).

ABTS.⁺ [2,2′-Azino-bis(3-ethylbenzothiazoline-6-sulfonic acid) diammonium salt] is a blue-colored radical cation which is reduced to colorless ABTS in a concentration-dependent manner upon addition of certain antioxidants that may be present in the TC/enriched tannoid blends prepared according to the principles of the present invention. This assay measures the capacity of an antioxidant sample to scavenge hydrophilic radicals. In the present study, antioxidant potentials of Terminalia chebula extracts were compared with trolox and the trolox equivalent antioxidant capacity (TEAC) value demonstrated the potential of the extracts as hydrophilic antioxidants.

Analytical Method.

ABTS stock solution was prepared by adding 88 μl of Ammonium persulphate solution (140 mM in water) to 5 ml of ABTS solution (7 mM in water) and keeping overnight. ABTS working solution was prepared by adding stock solution in phosphate buffered saline (PBS, pH 7.4) so that the absorbance of the solution becomes 0.8 at 734 nm. Test sample solution was prepared by dissolving sample in water at a concentration 0.1 mg/ml. Trolox was used as standard. Standard trolox solution was prepared by dissolving the substance in water at a concentration of 0.1 mg/ml. ABTS working solution was added to 20, 40, 60, 80 or 100 μl of sample and/or standard solution in a manner to get a final volume of 1000 μl. The mixture was kept at room temperature for 4 minutes and the absorbance were recorded at 734 nm. A control sample was prepared with PBS and without TC material. The experiment was conducted in duplicate.

Percent scavenging, as used herein, of the radical was calculated from the following formula, where OD=optical density.

$$Percent scavenging = \frac{{OD}_{control} - {OD}_{sample}}{{OD}_{control}} \qquad Equation ( 1 )$$

IC₅₀ values (concentration which scavenges 50% of the radical) were calculated by plotting percent scavenging on Y-axis and sample/standard concentration on X-axis. Results were expressed in “trolox equivalents.” Trolox equivalents were calculated from the following formula, where, X=IC₅₀ of trolox and Y=IC₅₀ of the sample.

$$Trolox equivalents = \frac{X}{Y} \times 4000 μmoles \text{/} g sample \qquad Equation ( 2 )$$

All the calculations of Trolox equivalents in the subsequent experiments were performed using the same formula.

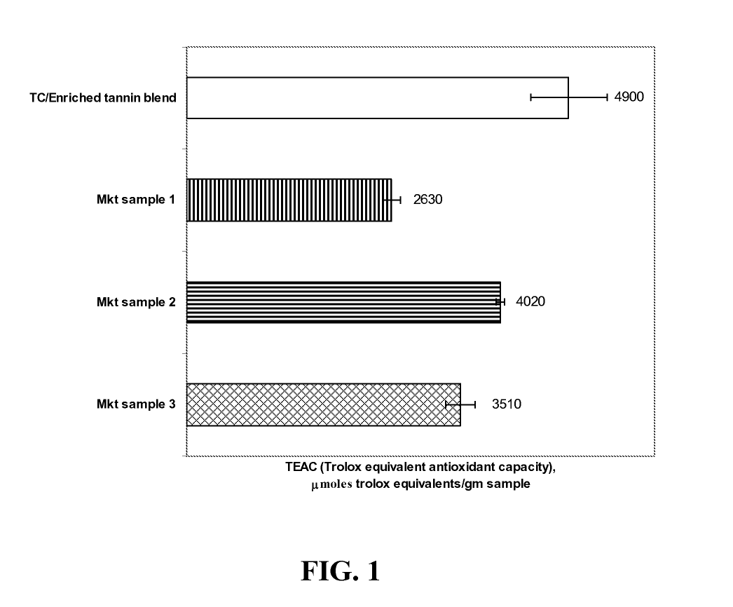

The results of the ABTS radical scavenging activity are depicted in FIG. 1. TC/Enriched tannoid blend exhibited the greatest Trolox equivalent antioxidant capacity (4900 mmoles/gm sample). The observed order of the four Terminalia chebula extracts for their ABTS radical scavenging capacities was: TC/Enriched tannoid blend>Mkt sample 2>Mkt sample 3>Mkt sample 1.

### Example 4B

DPPH radical decolorisation assay (I. Gulcin, et al., J. Ethnopharmacol. (2004) 90: 205-215).

DPPH (2,2-Diphenyl-1-picrylhydrazyl) is a purple-colored radical which is reduced to a colorless molecule in a concentration-dependent manner upon addition of certain antioxidants that may be present in the TC/enriched tannoid blends prepared according to the principles of the present invention. This assay measures the capacity of an antioxidant sample to scavenge lipophilic radicals.

Analytical Method.

Stock solution of 300 mM DPPH was prepared by dissolving 5.9 mg of DPPH in 50 ml ethanol and keeping the solution overnight. Test sample solution was prepared by dissolving samples in water at a concentration of 0.2 mg/ml. 25, 50, 75 and 100 μl of test sample solution were mixed with ethanol to get a final volume of 1000 μl. After vortexing, 0.5 ml of DPPH stock solution was added and incubated at room temperature for 20 minutes in diffused light. Readings were taken at 517 nm against blank. A control sample was prepared with ethanol and without TC material. The experiment was conducted in duplicate.

Percent scavenging of the radical was calculated as in Equation 1 above. IC₅₀ values (concentration which scavenges 50% of the radical) were calculated by plotting percent scavenging on Y-axis and sample concentration on X-axis. Results were expressed in IC₅₀ values. Since trolox did not show sensitivity in this experimental model, it was not used as standard for calculation of trolox equivalents.

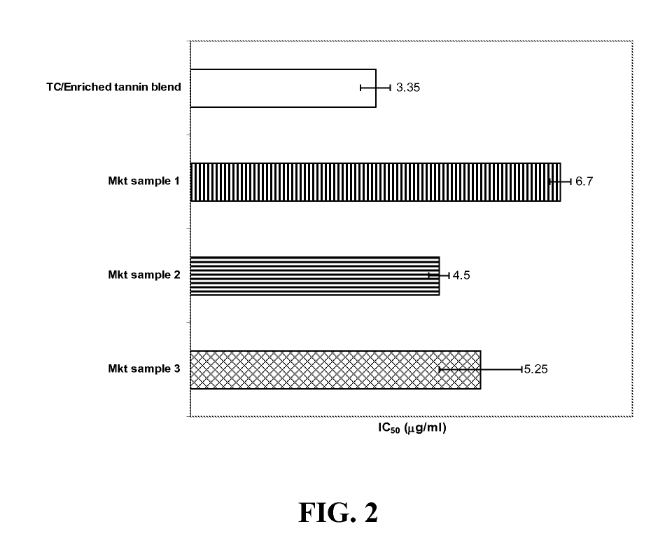

The results of the DPPH radical scavenging activity are depicted in FIG. 2. TC/Enriched tannoid blend exhibited the highest potency based on measured IC₅₀, which indicated higher antioxidant capacity. The observed order of the four Terminalia chebula extracts for their DPPH radical scavenging capacities was: TC/Enriched tannoid blend>Mkt sample 2>Mkt sample 3>Mkt sample 1.

In the present study, Terminalia chebula extracts demonstrated potential as lipophilic antioxidants in the assay protocol. The effect of the four extracts in the scavenging of DPPH radical furthermore assured the potential of the extracts as potent radical scavengers, since the studies of ABTS and DPPH radical scavenging assays as discussed above were found to be consistent and complementary to each other. The lower IC₅₀ values of TC/Enriched tannoid blend (3.35 μg/ml) compared to other known market samples indicated that the enriched tannoid blend was the most superior antioxidant among the four Terminalia chebula extracts.

### Example 4C

FRAP—Ferric reducing assay for plasma: Assay for reduction power (N. Pellegrini, et al., as above).

This assay is based upon reduction of ferric (Fe³⁺) ion by antioxidants to ferrous (Fe²⁺) ion and development of blue color by formation of a complex between Fe²⁺ and TPTZ (2,4,6-tri-(2-pyridyl)-s-triazine). The greater the electron donating power of the antioxidant, the greater the intensity of the blue color, which occurs in a concentration-dependent manner upon addition of certain antioxidants that may be present in the TC/enriched tannoid blends prepared according to the principles of the present invention.

Analytical Method.

FRAP reagent solution was prepared by mixing 1 ml of TPTZ [2,4,6-Tri(2-pyridyl)-s-triazine] solution (5 mM in TRIS-HCl), 10 ml of acetate buffer solution (100 mM in water, pH 3.9) and FeCl₃ solution (20 mM in water). FRAP reagent was prepared freshly each day before an experiment. Test sample solution was prepared by dissolving sample in water at a concentration 0.1 mg/ml. Trolox was used as standard. Standard trolox solution was prepared by dissolving the substance in water at a concentration of 0.1 mg/ml. FRAP reagent solution was added to 20, 40, 60 or 80 μl of sample and/or standard solution in a manner to get a final volume of 1000 μl and incubated for 30 minutes at 37° C. for reaction to take place. Then the absorbance was measured at 593 nm against reagent blank. A control sample devoid of TC material was also prepared. The experiment was conducted in duplicate.

Percent scavenging of the radical was calculated according to Equation 1 above, and Trolox equivalents (as TEAC) calculated according to Equation 2 as above.

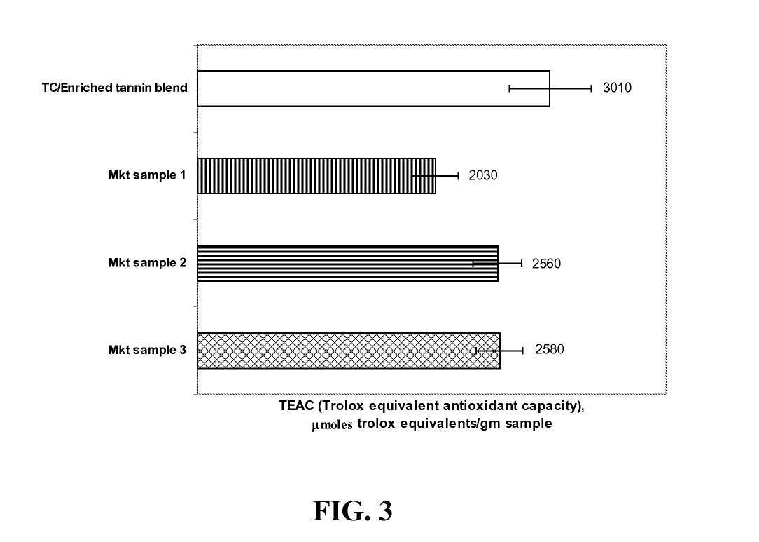

The results of the FRAP assay are depicted in FIG. 3. TC/Enriched tannoid blend exhibited the greatest Trolox equivalent antioxidant capacity (3010 μmoles/gm sample). The observed order of the four Terminalia chebula extracts for their reducing power was: TC/Enriched tannoid blend>Mkt sample 3>Mkt sample 2>Mkt sample 1.

Thus, the reducing power of Terminalia chebula extracts, as shown in the above studies, may serve as a significant indicator of their potential electron donating capacity. The reducing power of the four extracts showed considerable activities, which were also in accordance with their radical scavenging assays. The result also indicated that the enriched tannoid blend was the best among the four Terminalia chebula extracts.

### Example 4D

Hydroxyl radical scavenging assay by 2-deoxyribose degradation method (S. K. Chung, et al., Biosci. Biotech. Biochem. (1997) 61: 118-123).

Hydroxyl radical may be the most deleterious radical present in the physiological systems, which can damage almost all biomolecules, including DNA. For this reason, hydroxyl radical scavenging potential of an extract may be ascertained to estimate the effectiveness of an extract as a potent antioxidant. The model used to measure the hydroxyl radical scavenging abilities of the Terminalia chebula extracts in this study is based upon Fenton's reaction, as certain antioxidants may be present in the TC/enriched tannoid blends prepared according to the principles of the present invention. As the extracts or the standard were added to the solution containing Fenton's reaction mixture, the hydroxyl radicals were scavenged thereby blocking the formation of MDA (Malondialdehyde), which was generated by degradation of the sugar, 2-deoxyribose.

Analytical Method.

Test sample solution was prepared by dissolving sample in distilled water at a concentration 10 mg/ml. 50, 100, 150 or 200 μl of sample solution were added to 200 μl of each of FeSO₄.7H₂O solution (10 mM in water) EDTA solution (10 mM in water), 2-deoxyribose solution (10 mM in water) and H₂O₂ (10 mM in water) to get a final volume of 1000 μl. Remaining volume was made up with water. Then the mixtures were incubated for 1 hour at 37° C. After incubation, 500 μl of TCA solution (10% w/v, in water) and 500 μl of TBA solution (1.2% w/v, in water) were added and warmed at 90° C. for 10 minutes. The absorbance was read at 532 nm against reagent blank after cooling of the mixtures to room temperature. A control sample was prepared with water and without TC material. The experiment was conducted in duplicate.

Percent scavenging of the radical was calculated according to Equation 1 above, and Trolox equivalents (as TEAC) calculated according to Equation 2 as above.

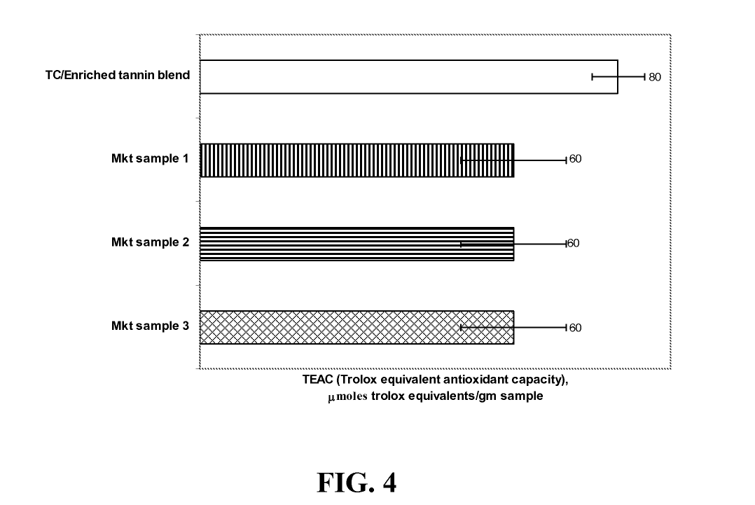

The results of the hydroxyl radical scavenging assay are depicted in FIG. 4. TC/Enriched tannoid blend exhibited the greatest Trolox equivalent antioxidant capacity (80 mmoles/gm sample). The observed order of the four Terminalia chebula extracts for their OH. radical scavenging capacities was: TC/Enriched tannoid blend>Mkt sample 2≈Mkt sample 3≈Mkt sample 1. (The observed results obtained from the market samples were substantially the same.) The result also indicated that the enriched tannoid blend provided the best radical scavenging capacity from among the four Terminalia chebula extracts.

### Example 4E

Superoxide radical scavenging assay by NBT (Nitro Blue TetraZolium)-NADH (Nicotinamide Adenine Dinucleotide-Reduced)-PMS (Phenazonium Methosulphate) method (I. Gulcin, et al., J. Ethnopharmacol. (2004) 90: 205-215, as above).

Superoxide radicals are generated in the mitochondria as useful molecules, but at very low concentrations. At high concentrations, they are potentially as deleterious as hydroxyl radicals. The generated radicals were tested along with certain antioxidants that may be present in the TC/enriched tannoid blends prepared according to the principles of the present invention, in order to assess antioxidant capacities.

Analytical Method.

Test sample solution was prepared by dissolving sample in distilled water at a concentration 1 mg/ml. 20, 40, 60 or 80 μl of sample solution were mixed with 500 μl of each of NADH solution (300 μM in TRIS-HCl), NBT solution (100 μM in TRIS-HCl) and PMS solution (20 μM in TRIS-HCl) to provide a final volume of 2000 μl. Remaining volume was made up with TRIS-HCl. The mixture was incubated for 30 minutes at room temperature for reaction to take place. Then the absorbance of the blue formazan, developed during reaction, was measured at 560 nm against reagent blank. A control sample was prepared with water and without TC material. The experiment was conducted in duplicate.

Percent scavenging of the radical was calculated as in Equation 1 above. IC₅₀ values (concentration which scavenges 50% of the radical) were calculated by plotting percent scavenging on Y-axis and sample concentration on X-axis. Results were expressed in IC₅₀ values. Since trolox did not show sensitivity in this experimental model, it was not used as standard for calculation of trolox equivalents.

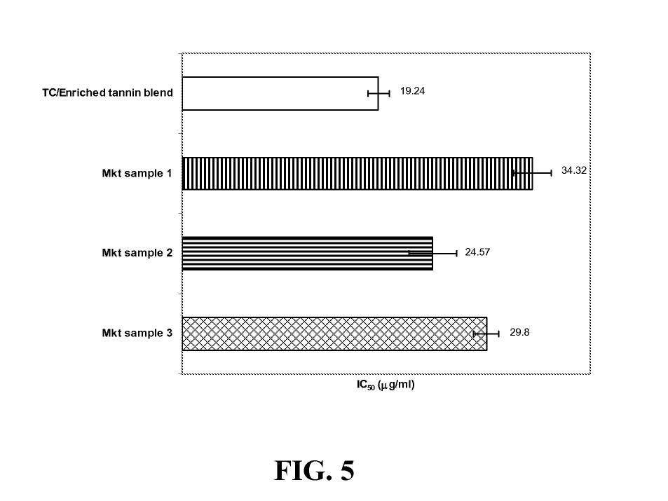

As shown in FIG. 5, the present study showed the abilities of the four Terminalia chebula extracts to quench superoxide (O₂.⁻) radicals generated from PMS-NADH reaction mixture. The results indicated that the extracts were good quenchers of O₂.⁻ radicals. The observed order of the four Terminalia chebula extracts for their O₂.⁻ radical scavenging capacities was: TC/Enriched tannoid blend>Mkt sample 2>Mkt sample 3>Mkt sample 1. The result thus indicated that the TC/enriched tannoid blend was the most potent antioxidant among the four Terminalia chebula extracts.

### Example 4F

Hydrogen peroxide scavenging assay (I. Gulcin, et al., as above).

Hydrogen peroxide is a weak oxidizing agent and can inactivate a few enzymes directly, usually by oxidation of essential thiol groups. It can also cross the cell-membrane rapidly. Once inside the cell, the compound can react with Fe²⁺ and Cu²⁺ ions to form hydroxyl radical, which potentially has immense toxic effects.

As certain antioxidants may be present in the TC/enriched tannoid blends prepared according to the principles of the present invention, a model was used to measure the peroxide scavenging abilities of the Terminalia chebula extracts in this study.

Analytical Method.

A stock of 40 mM H₂O₂ solution in 100 mM phosphate buffer (pH 7.4) was prepared for the experiment. Test sample solution was prepared by dissolving sample in water at a concentration 2 mg/ml. Trolox was used as standard. Standard trolox solution was prepared by dissolving the substance in water at a concentration of 1 mg/ml. Stock H₂O₂ solution was added to 25, 50, 75 or 100 μl of sample and/or standard solution in a manner to get a final volume of 1000 μl. The mixture was incubated for 30 minutes at room temperature and the absorbance was recorded at 230 nm against reagent blank. Reaction mixture with sample at 0 minute was used as control. The experiment was conducted in duplicate.

Percent scavenging of H₂O₂ was calculated according to Equation 1 above. IC₅₀ values (concentration which scavenges 50% of the oxidant) were calculated by plotting percent scavenging on Y-axis and sample/standard concentration on X-axis. Trolox equivalents (as TEAC) were calculated according to Equation 2 as above.

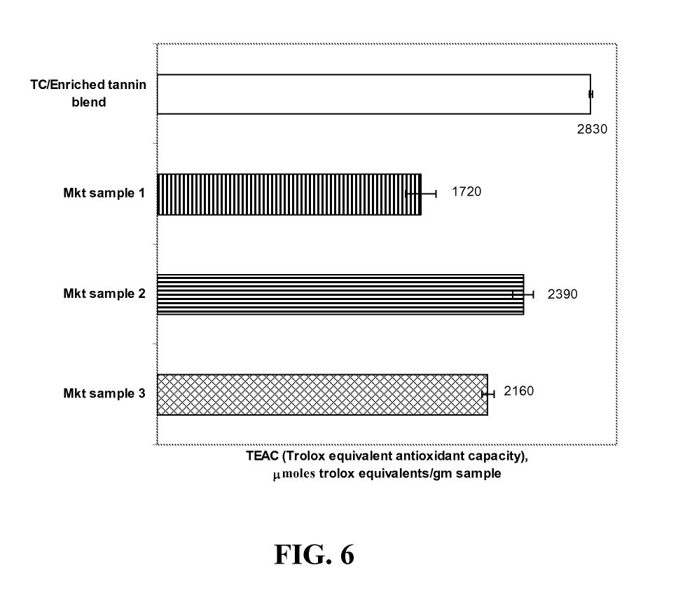

As shown in FIG. 6, the present study showed the abilities of the four Terminalia chebula extracts to scavenge H₂O₂. The results indicated that the extracts were good scavengers of the reactive oxygen species. The result also indicated that the enriched TC/tannoid blend possessed the greatest antioxidant capacity among the four Terminalia chebula extracts. The observed order of the four Terminalia chebula extracts for their H₂O₂ scavenging capacities was: TC/Enriched tannoid blend>Mkt sample 2>Mkt sample 3>Mkt sample 1, indicating potential of the extracts for scavenging of H₂O₂.

### Example 4G

Estimation of total phenolic content by Folin-Ciocalteu method (S. A. L. Morais, et al., J. Braz. Chem. Soc. (1999) 10:447-452).

Total phenolic content is an important parameter for the determination of antioxidant potential of natural product extracts as it denotes their reducing power due to the presence of active hydrogen in the phenolic molecules present in the antioxidant extracts. The model is used to measure the total phenolic content of the Terminalia chebula extracts in this study, as certain phenolic or polyphenolic antioxidants may be present in the TC/enriched tannoid blends prepared according to the principles of the present invention.

Analytical Method.

Test sample solution was prepared by dissolving sample in water at a concentration of 1 mg/ml. Gallic acid was used as standard. Standard gallic acid solution was prepared by dissolving the substance in water at a concentration of 1 mg/ml. For the preparation of gallic acid standard curve, 5, 10, 15 or 20 μA of the solution was mixed with 100 μl Folin-Ciocalteu working reagent (commercial Folin-Ciocalteu reagent diluted 10-fold with distilled water) in a manner to provide a final volume of 500 μl by adding distilled water. The mixtures were kept at room temperature for 5 minutes. Then 500 μl of 7.5% Na₂CO₃ solution (w/v, in water) was added and incubated at 50° C. for 5 minutes. After incubation, the absorbance was recorded at 740 nm against reagent blank. An absorbance vs. gallic acid concentration curve was prepared by plotting absorbance on Y-axis and sample concentration on X-axis. For test sample solutions, 10, 20, 30 and 40 μl were added separately in Folin-Ciocalteu working reagent followed by Na₂CO₃ solution to obtain four different absorbance vs. concentration values. By putting the absorbance values in the gallic acid standard curve, gallic acid equivalents of the samples were obtained.

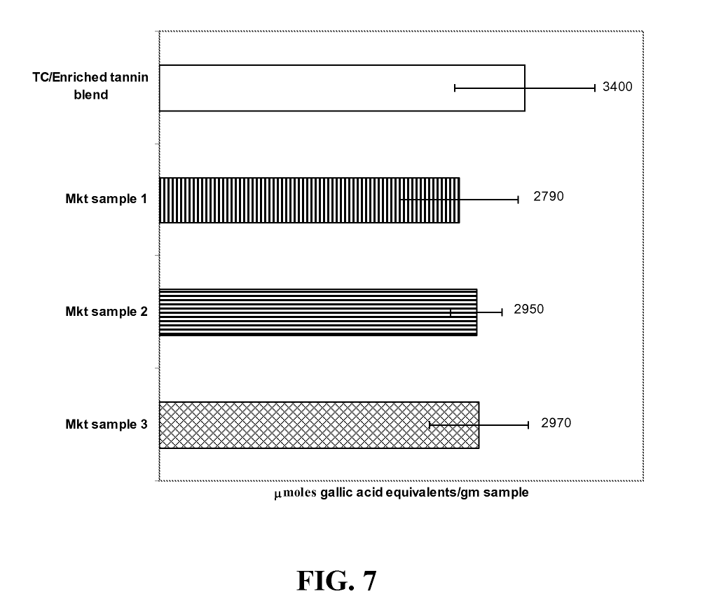

As demonstrated in FIG. 7, the results of the total phenolic contents (expressed in gallic acid equivalents) of the four Terminalia chebula extracts indicate presence of significant amounts of phenolics in the samples. The result also indicated that the TC/enriched tannoid blend possessed the greatest levels among the four Terminalia chebula extracts with respect to their phenolic contents. The observed order of the four Terminalia chebula extracts for their total phenolic contents was: TC/Enriched tannoid blend >Mkt sample 3>Mkt sample 2>Mkt sample 1.

### Example 4H

Nitric oxide radical scavenging assay (R. Sundararajan, et al., Complementary & Alternative Medicine (2006) δ: 8-14).

Nitric oxide (NO) is an essential biomolecule, which serves as a neurotransmitter in the human body at a very low concentration. However, it may play deleterious roles, when in excess, for example, in various types of inflammatory conditions including juvenile diabetes, multiple sclerosis, arthritis and ulcerative colitis. In the present study, NO generated from sodium nitroprusside reacts with oxygen to form nitrite anion, which was well restrained by the antioxidants, including certain antioxidants that may be present in the TC/enriched tannoid blends prepared according to the principles of the present invention. The NO scavenging property was monitored by reacting the nitrite with aromatic nitrogen compound, NDDCL to form an azo dye.

Analytical Method.

Test sample solution was prepared by dissolving sample in phosphate buffered saline (PBS, pH 7.4) at a concentration 1 mg/ml. 10, 20, 30 and 40 μl of sample solution were mixed with 2000 μl sodium nitroprusside solution (5 mM in water, source of NO radical) and required volume of PBS to get a final volume of 2500 μA. The mixture was incubated at room temperature for 150 minutes. 500 μl of aliquot was taken and mixed with 500 μl of a 1:1 (v/v) mixture of 1% sulphanilic acid (in 20% glacial acetic acid) and 2% ortho-phosphoric acid (in 20% glacial acetic acid). After 5 minutes incubation, 250 μl of 0.1% NDDCL reagent solution (N-1-napthyl-ethylenediamine dihydrochloride, in 20% glacial acetic acid) was added and allowed to incubate for 30 minutes in diffused light at room temperature. The absorbance after incubation was measured at 540 nm against reagent blank. Trolox was used as standard. Standard trolox solution was prepared by dissolving the substance in water at a concentration of 1 mg/ml. In case of determination of IC₅₀ of trolox, 5, 10, 15 and 20 μl of the solution were used. The experiment was performed in duplicate.

Percent scavenging of the NO radical was calculated according to Equation 1 above, and Trolox equivalents (as TEAC) calculated according to Equation 2 as above.

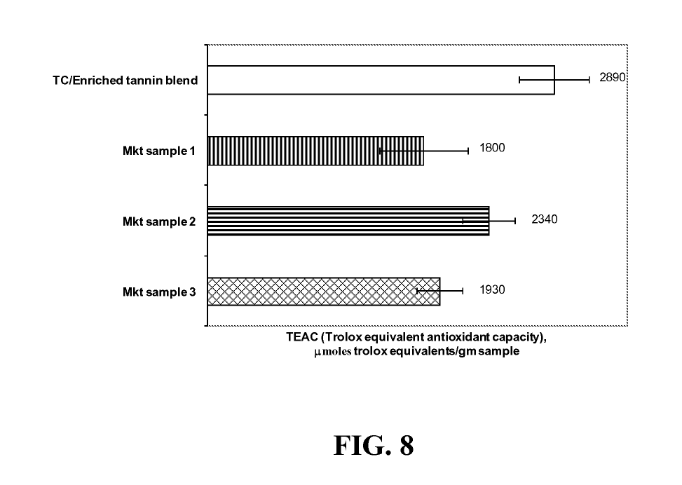

As shown in FIG. 8, the results of the four Terminalia chebula extracts thus indicate significant NO scavenging activities in the test protocol. The observed order of the four Terminalia chebula extracts for their NO radical scavenging capacities was: TC/Enriched tannoid blend>Mkt sample 2>Mkt sample 3>Mkt sample 1. The result thus indicated that the TC/enriched tannoid blend possessed the greatest antioxidant capacity among the four Terminalia chebula extracts.

### Example 4I

Peroxynitrite scavenging assay (L. B. Valdez, et al., Medicina (Lithuania), (2007) 43: 306-309).

The potential lethal consequence of nitric oxide increases significantly upon reaction with superoxide radicals, resulting in the formation of highly reactive peroxynitrite anion (ONOO⁻). This radical has been implicated in the pathogenesis of diseases such as heart disease, Alzheimer's disease and atherosclerosis. The generated radicals were tested along with certain antioxidants that may be present in the TC/enriched tannoid blends prepared according to the principles of the present invention, in order to assess antioxidant capacities.

Analytical Method.

Stock peroxynitrite anion solution was prepared by reacting 50 ml of H₂O₂ (2M in 2M HNO₃) and 50 ml of NaNO₂ solution (2M in water) at 0° C. for 30 minutes followed by stabilization of the product with 50 ml of NaOH solution (4M in water). The solution was kept at −20° C. overnight. A yellow colored upper layer separated over the ice was used as stock peroxynitrite solution. Peroxynitrite concentration was determined spectrophotometrically at 302 nm and dilutions were made with 1M NaOH solution so as to achieve absorbance of the peroxynitrite working solution of 0.7-0.8. Test sample solution was prepared by dissolving sample in distilled water at a concentration 10 mg/ml. 50, 100, 150 and 200 μl of test sample solution were added to peroxynitrite working solution to achieve a final volume of 1000 μl. The mixture was incubated for 30 minutes at room temperature and the absorbance was recorded at 302 nm against reagent blank. Reaction mixture with sample at 0 minute was used as control. The experiment was conducted in duplicate.

Percent scavenging of the peroxynitrite radical was calculated according to Equation 1 above. IC₅₀ values (concentration which scavenges 50% of the anion) were calculated by plotting percent scavenging on Y-axis and sample/standard concentration on X-axis. Results were expressed in “ascorbic acid equivalents.” Ascorbic acid equivalents were calculated from the following formula, where X=IC₅₀ of ascorbic acid and Y=IC₅₀ of the sample.

$$Ascorbic acid equivalents = \frac{X}{Y} \times 5690 μmoles \text{/} g {sample}^{*} \\ {}^{*} [ since mol . weight of ascorbic acid is 176 ] \qquad Equation ( 3 )$$

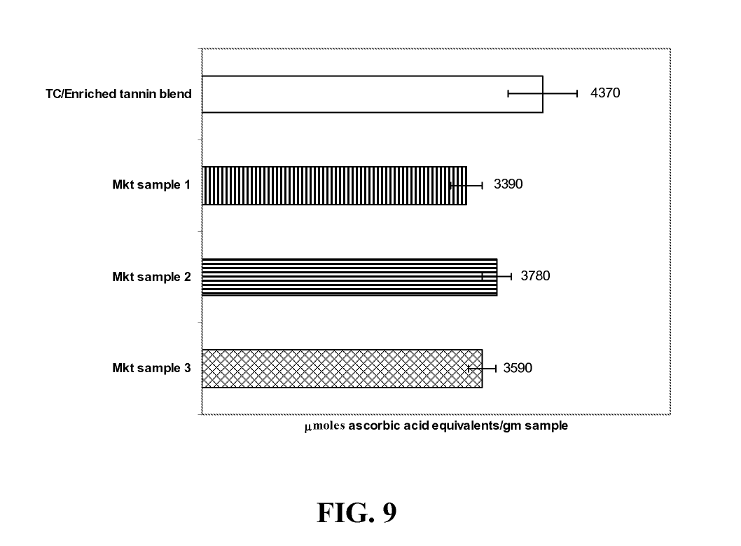

As shown in FIG. 9, the results demonstrated that peroxynitrite anion can be effectively scavenged by the Terminalia chebula extracts. The observed order was found to be: TC/Enriched tannoid blend>Mkt sample 2>Mkt sample 3>Mkt sample 1. The result thus indicated that the TC/enriched tannoid blend possessed the greatest antioxidant capacity among the four Terminalia chebula extracts.

### Inhibition of Lipid Peroxidation

### Examples 4J and 4K

It has been known that polyunsaturated fatty acids (PUFA) are more susceptible to free radical assault, whereas monounsaturated or saturated fatty acids are generally resistant to radical attacks. This indicates that antioxidant extracts might behave differently against different lipid compositions and their activity might not be the same against lipid peroxidation in vivo, as different parts of the body contain lipids having different compositions. In the present study, two models of inhibition of lipid peroxidation were used (viz. erythrocyte membrane lipid peroxidation and goat-brain lipid peroxidation). The lipid sources were prepared differently in the two assays so as to adjudicate the capabilities of the antioxidant extracts in terminating chain reaction in two different types of lipids, containing different proportions of PUFA and saturated fatty acids, as follows.

As certain antioxidants may be present in the TC/enriched tannoid blends prepared according to the principles of the present invention, the studies were used to measure the antioxidant capacities of the Terminalia chebula extracts.

### Example 4J

Inhibition of rat erythrocyte membrane lipid peroxidation (T. Moriguchi, et al., J. Nutr. (2001) 131: 1016S-1019S).

Analytical Method.

For this experiment, TBA (2-thiobarbituric acid) reagent was prepared by dissolving 375 mg of TBA (2-thiobarbituric acid), 15 gms of TCA (trichloroacetic acid) and 400 mg of BHT (butylated hydroxytoluene) in 1 L of 1M aqueous HCl. Fresh rat blood was centrifuged at 2500 rpm for 5 minutes to separate erythrocytes. After removal of plasma, the erythrocyte suspension was washed with 10 volumes of PBS (phosphate buffered saline, pH 7.4) three times. Then the erythrocytes (1 volume) were suspended in PBS (10 volumes) to adjust the hematocrit of 10%. Before actual work, the erythrocyte suspension (ES) was pre-incubated at 37° C. for 30 minutes. Test sample solution was prepared by dissolving sample in PBS (pH 7.4) at a concentration 10 mg/ml. 50, 100, 150 and 200 μl of sample solution were added to ES (500 μl) followed by 150 μl of ascorbic acid solution (1 mM in water) and 150 μl of FeSO₄.7H₂O solution (5 mM in water) to provide a final volume of 1000 μl. Remaining volume was made up with PBS. Trolox was used as standard. Standard trolox solution was prepared by dissolving the substance in water at a concentration of 10 mg/ml. In case of determination of IC₅₀ of trolox, 25, 50, 75 and 100 μl of the solution were used. The mixtures were incubated for 3 hours at 37° C. Then 1000 μl of TBA reagent solution was added and warmed at 90° C. for 15 minutes. The mixture was cooled to room temperature and centrifuged at 2500 rpm for 15 minutes. The absorbance values were read at 535 nm against reagent blank. The experiment was performed in duplicate.

Percent inhibition, as used herein, of lipid peroxidation was calculated from the following formula, where OD=optical density.

$$Percent inhibition = \frac{{OD}_{control} - {OD}_{sample}}{{OD}_{control}} \times 100 \qquad Equation ( 4 )$$

IC₅₀ values (concentration which inhibits 50% of peroxidation) were calculated by plotting percent inhibition on Y-axis and sample/standard concentration on X-axis. Results were expressed in “trolox equivalents.” Trolox equivalents (as TEAC) were calculated according to Equation 2 as above.

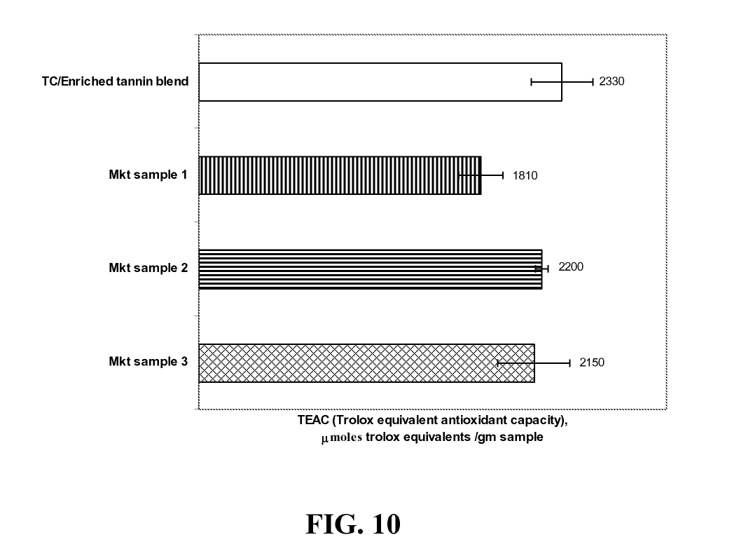

As shown in FIG. 10, the results indicated that the extracts were good terminators of chain reaction which leads to lipid peroxidation. In erythrocyte membrane lipid peroxidation model, the observed order of the activity of four Terminalia chebula extracts was found to be: TC/Enriched tannoid blend>Mkt sample 2>Mkt sample 3>Mkt sample 1. The result thus indicated that the TC/enriched tannoid blend possessed the greatest antioxidant capacity among the four Terminalia chebula extracts.

### Example 4K

Inhibition of lipid peroxidation of brain homogenate (S. K. Mondal, et al., Ind. J. Exp. Biol. (2006) 44: 39-44).

Analytical Method.

For this experiment, TBA reagent was prepared by dissolving 375 mg of TBA, 15 μms of TCA and 400 mg of BHT in 1 L of 1M aqueous HCl. Goat brain homogenate was prepared by homogenizing 10 gms of fresh goat brain in 100 ml PBS (pH 7.4) and separating the supernatant by cold-centrifugation (10,000 rpm, 15 minutes). The supernatant, rich in lipid, was pre-incubated at 37° C. for 30 minutes before actual work. Test sample solution was prepared by dissolving sample in PBS (pH 7.4) at a concentration 10 mg/ml. 5, 10, 15 and 20 μl of sample solution were added to the supernatant (500 μl) followed by 150 μl of ascorbic acid solution (1 mM in water) and 150 μl of FeSO₄.7H₂O solution (5 mM in water). Remaining volume was made up with PBS to 1000 μl. The mixtures were incubated for 3 hours at 37° C. Then 1000 μl of TBA reagent solution was added and warmed at 90° C. for 15 minutes. The mixture was cooled to room temperature and centrifuged at 2500 rpm for 15 minutes. The absorbance values were read at 535 nm against reagent blank. The experiment was performed in duplicate.

Percent inhibition of lipid peroxidation was calculated according to Equation 4 above, and Trolox equivalents (as TEAC) were calculated according to Equation 2 as above.

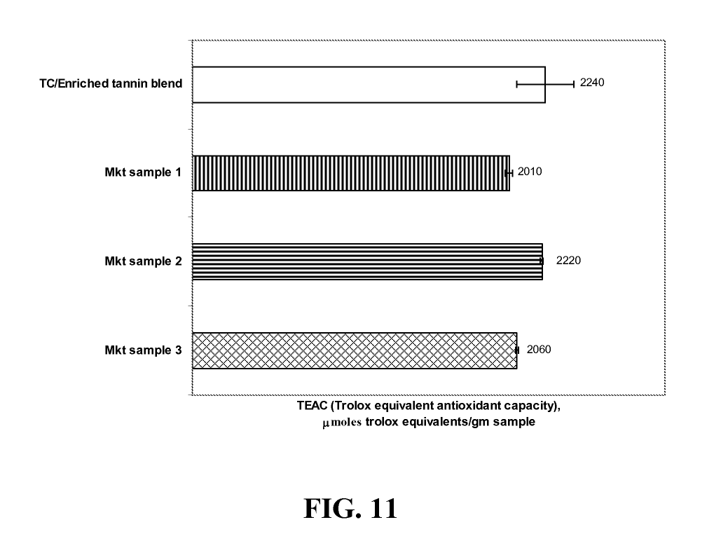

As shown in FIG. 11, the results indicated that the extracts were good terminators of the chain reaction which leads to lipid peroxidation. In this lipid peroxidation model, the observed order of the activity of four Terminalia chebula extracts was found to be: TC/Enriched tannoid blend>Mkt sample 2>Mkt sample 3>Mkt sample 1. The result thus indicated that the TC/enriched tannoid blend possessed the greatest antioxidant capacity among the four Terminalia chebula extracts.

In conclusion, the results of the study demonstrated that all the Terminalia chebula extracts showed significant antioxidant activity against different free radicals, e.g., hydroxyl, superoxide, hydrogen peroxide, nitric oxide, peroxynitrite radicals, and the like. However, the extract of the TC/enriched tannoid blend showed much better antioxidant activity, in all the models tested, than those extracts of Market Samples 1-3, demonstrating the superior properties of the TC/enriched tannoid blend composition.

It is further expected that a hydrolyzable tannoid enriched T. chebula extract made in accordance with the principles of the invention would be effective as a nutritional supplement.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A Terminalia chebula extract composition comprising a hydrolyzable tannoid blend including about 8-25% by weight chebulagic acid based on the total weight of the extract, and about 15-30% by weight chebulinic acid based on the total weight of the extract.

2. The Terminalia chebula extract composition of claim 1, further comprising about 5-45% by weight other low molecular weight hydrolyzable tannoids based on the total weight of the extract.

3. A pharmaceutical, nutraceutical, or skin care composition comprising the extract of claim 1 and a pharmaceutically, nutraceutically, or cosmeceutically acceptable carrier.

4. The Terminalia chebula extract composition of claim 1, wherein chebulagic acid is present in an amount of about 15-20% by weight based on the total weight of the extract, and chebulinic acid is present in an amount of about 20-28% by weight based on the total weight of the extract.

5. A method of making a Terminalia chebula extract composition comprising a hydrolyzable tannoid blend, including the steps of:
(a) providing fruit pericarp portions of a Terminalia chebula plant;
(b) grinding the fruit pericarp Terminalia chebula plant portions to provide a powder;
(c) extracting the Terminalia chebula fruit pericarp powder with water to provide a Terminalia chebula aqueous extract; and
(d) drying the Terminalia chebula aqueous extract to provide a Terminalia chebula extract as a powder.

6. The method of claim 5, wherein the drying step is carried out by spray-drying or freeze drying.

7. The method of claim 5, wherein the Terminalia chebula extract powder comprises a hydrolyzable tannoid blend including about 8-25% by weight chebulagic acid based on the total weight of the extract powder, and about 15-30% by weight chebulinic acid based on the total weight of the extract powder.

8. The method of claim 7, wherein the Terminalia chebula extract powder further comprises about 5-45% by weight other low molecular weight hydrolyzable tannoids based on the total weight of the extract powder.

9. The method of claim 7, wherein chebulagic acid is present in an amount of about 15-20% by weight based on the total weight of the extract powder, and chebulinic acid is present in an amount of about 20-28% by weight based on the total weight of the extract powder.

10. The method of claim 7, wherein the extracting step is carried out at about 25° C. for a time of about 12 hours to about 16 hours.

11. The method of claim 7, wherein the extracting step is carried out at about 40° C. for a time of about 1 hour to about 6 hours.

12. The method of claim 7, wherein the extracting step is carried out at about 40° C. for a time of about 3 hours.

13. A method of treating or preventing a free radical-induced disease or ailment in an individual, comprising administering to the individual in need of such treatment a therapeutically effective amount of a composition according to claim 1, wherein the Trolox equivalent antioxidant capacity (TEAC) of the composition is at least about 2000 μmoles of trolox equivalents/gm.

14. The method of claim 13, wherein the Trolox equivalent antioxidant capacity (TEAC) of the composition is at least about 3000 μmoles of trolox equivalents/gm.

15. The method of claim 13, wherein the Trolox equivalent antioxidant capacity (TEAC) of the composition is at least about 4000 μmoles of trolox equivalents/gm.

16. A method of treating or preventing a free radical-induced disease or ailment in an individual, comprising administering to the individual in need of such treatment a therapeutically effective amount of a composition according to claim 3, wherein the Trolox equivalent antioxidant capacity (TEAC) of the composition is at least about 2000 μmoles of trolox equivalents/gm.

17. The method of claim 16, wherein the Trolox equivalent antioxidant capacity (TEAC) of the composition is at least about 3000 μmoles of trolox equivalents/gm.

18. The method of claim 16, wherein the Trolox equivalent antioxidant capacity (TEAC) of the composition is at least about 4000 μmoles of trolox equivalents/gm.
